(19) United States
(12) Patent Application Publication
(10) Pub. No.: US 2005/0014955 A1
(43) Pub. Date: Jan. 20, 2005

(54) Process for the preapartion 3-aryl-2-hydroxypropionic acid derivative

(76) Inventors: Robert Ehrl, Sodetalje (SE); William Mackintosh, Sodetalje (SE); Panagiotis Ioannidis, Spanga (SE)

Correspondence Address: ROPES & GRAY LLP, ONE INTERNATIONAL PLACE, BOSTON, MA, 02110-2624, US

(21) Appl. No.: 10/479,159

(22) PCT Filed: May 30, 2002
(86) PCT No.: PCT/SE02/01040

(30) Foreign Application Priority Data
Jun. 1, 2001 (SE) 0101979-3
Apr. 2, 2002 (SE) 0201004-9

(51) Int. Cl. C07D 207/46; C 07C 3 3/40

(52) U.S. Cl. 548/577; 558/52

(57) ABSTRACT

A process for the preparation of a compound of formula I 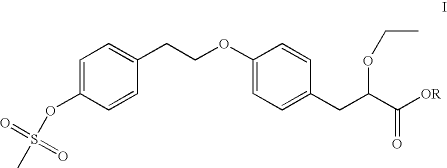
in which R represents H or an acid protecting group which comprises reacting a compound of formula II 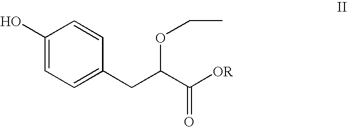
in which R is as previously defined with a compound of formula III 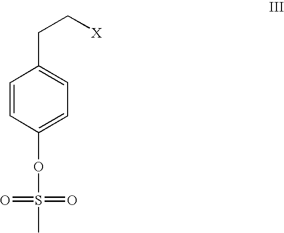 wherein X is a suitable leaving group in the presence of a base and a phase transfer catalyst at a temperature in the range 50° C. to 150° C.

### RELATED APPLICATIONS

This application is a national stage filing under 35 U.S.C. 371 of International Application PCT/SE02/01040, filed May 30, 2002, which claims priority from Swedish Applications Nos. 0101979-3, filed June 1, 2001, and 0201004-9, filed Apr. 2, 2002, the specifications of which are incorporated by reference herein. International Application PCT/SE02/01040 was published under PCT Article 21(2) in English.

The present invention relates to an improved process for the preparation of the compound 2-ethoxy-3-[4-(2-{4-methanesulfonyloxyphenyl}ethoxy)phenyl]propanoic acid, as shown in formula I below 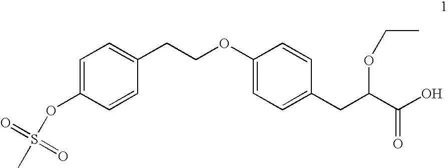
or the (R) or the (S) enantiomer thereof, or a pharmaceutically-acceptable salt thereof, and solvates thereof.

The above compound is intended for therapeutic use in the Insulin Resistance Syndrome (IRS) including type 2 diabetes mellitus, which refers to a cluster of manifestations including insulin resistance with accompanying hyperinsulinaemia, possible type 2 diabetes mellitus, arterial hypertension, central (visceral) obesity, dyslipidaemia observed as deranged lipoprotein levels typically characterised by elevated VLDL (very low density lipoproteins), small dense LDL particles and reduced HDL (high density lipoprotein) concentrations and reduced fibrinolysis.

Recent epidemiological research has documented that individuals with insulin resistance run a greatly increased risk of cardiovascular morbidity and mortality, notably suffering from myocardial infarction and stroke. In type 2 diabetes mellitus atherosclerosis related conditions cause up to 80% of all deaths.

In clinical medicine there is awareness of the need to increase the insulin sensitivity in IRS suffering patients and thus to correct the dyslipidaemia which is considered to cause the accelerated progress of atherosclerosis. However, currently this is not a universally well defined disease.

The compound of formula I is disclosed in PCT Publication Number WO99/62872. Two alternative processes are disclosed for the preparation of the compound of formula I in Examples 1 and 2 of the application. We have discovered an improvement in relation to one of the processes disclosed.

Specifically we have found an improved method for the synthesis of the penultimate esterified intermediate, the final step of the reaction being the conversion of the ester group into the acid of the final product.

Accordingly the present invention provides a process for the preparation of a compound of formula I 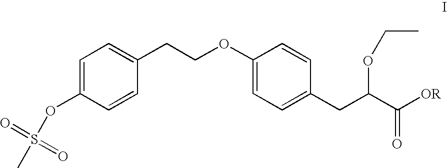
in which R represents H or an acid protecting group which comprises reacting a compound of formula II 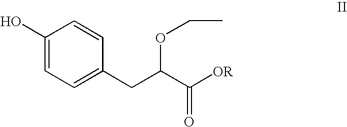
in which R is as previously defined with a compound of formula III 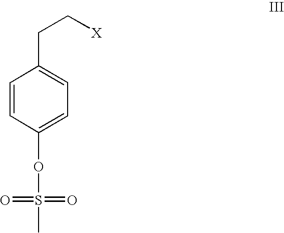
wherein X is a suitable leaving group in the presence of a base and a phase transfer catalyst at a temperature in the range 50° C. to 150° C.

In another aspect the present invention provides a process for the preparation of a compound of formula I 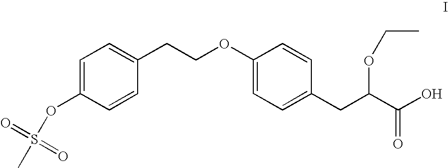
in which R represents H or an acid protecting group which comprises reacting a compound of formula II 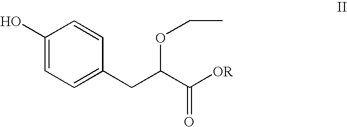
in which R is as previously defined with a compound of formula III 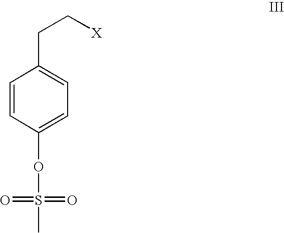
wherein X is a suitable leaving group in the presence of an aqueous solution of a base and a phase transfer catalyst at a temperature in the range 50° C. to 150° C.

In another aspect the present invention provides a process for the preparation of a compound of formula I 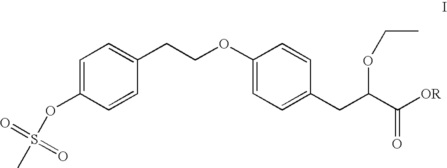
in which R represents H or an acid protecting group which comprises reacting a compound of formula II 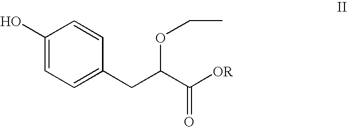
in which R is as previously defined with a compound of formula III 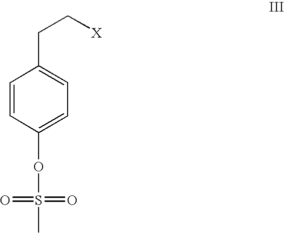
wherein X is a suitable leaving group in the presence of a base in solid form and a phase transfer catalyst at a temperature in the range 50° C. to 150° C.

The process may be carried out in a melt or in the presence of a suitable solvent for the compounds of formulae II and III. Preferably the process is carried out at a temperature in the range of 80° C. to 130° C. and most preferably in the range of 90° C. to 110° C.

The term “acid protecting group” means that the acid is protected from reaction by forming a suitable acid derivative such as an ester or amide or by other means of protection of carboxylic acid groups known in the art. Examples of suitable means of protection and acid derivatives (as well as means of formation and eventual deprotection), may be found in T. W. Greene and P. G. M. Wuts, “Protective Groups in Organic Synthesis”, Third Edition, John Wiley & Sons, New York, 1999. The nature of the ester is not important in the performance of the process since its function is to act as a protecting group. The improvements relate to the application of phase transfer catalysis to the process. Preferably R is H, benzyl or a (1-4C)alkyl group, such as methyl, ethyl or propyl. More preferably R is a (1-4C)alkyl group. Most preferably R is ethyl.

Each of the aforementioned processes may also comprise an additional step in which the the protecting group is removed to produce a compound of formula I in which R is H. Preferably R is an ester and the protecting group removal step comprises a hydrolysis step. The hydrolysis step may be acid or base catalysed (for example using lithium hydroxide). Optionally an organic liquid may be present in the hydrolysis step for example acetone, 2-butanone, methanol, ethanol, tetrahydrofuran or dioxane. Converting the acid ester derivative may be accomplished simply by hydrolysis (acidic or alkaline or enzymatic) of the ester to the acid, such a step being known to the skilled person, such as described in the examples below and in Example 2 i) of WO99/62872.

Suitably X is halo, for example bromo, chloro or iodo, an optionally substituted phenylsulfonyloxy group, in particular (4-methylphenyl)sulfonyloxy group or 2,4,6-triisopropylphenylsulfonyloxy group or an alkylsulphonyloxy group for example methanesulphonyloxy. Preferably X is a methanesulphonyloxy group.

Suitable bases include carbonates, hydrogen carbonates or hydroxides particularly of alkali metals. Preferably the base is sodium carbonate, sodium hydrogen carbonate, potassium carbonate or potassium hydrogen carbonate.

Suitably the phase transfer catalyst is a crown ether, a polyethylene glycol or a quaternary ammonium salt particularly with a halide counterion. Suitable crown ethers include 18 crown 6, dicyclohexyl[18-crown-6] and dibenzo[18-crown-6]. Suitable polyethylene glycols include PEG 400. Suitable quaternary ammonium salts include tetrahexylammonium bromide, methyltrioctylammonium chloride and tetraoctylammonium bromide.

Suitably the molar ratio of the compound of formula III to the compound of formula II is in the range of 0.5 to 10, preferably in the range 0.8 to 4 and more preferably in the range of 1.0 to 3 and most preferably is in the range of 1.2 to 1.6.

Suitably the weight ratio of the phase transfer catalyst to the compound of formula II is in the range of 0.05 to 10, preferably in the range 0.1 to 5 and more preferably in the range of 0.15 to 3.

Suitably the molar ratio of the base to the compound of formula II is in the range of 0.5 to 10, preferably in the range 0.8 to 4 and more preferably in the range of 1.0 to 3 and most preferably is in the range of 1.2 to 1.6.

The solvent, if used, is an organic solvent. The organic solvent may be either a protic or an aprotic solvent, preferably an aprotic solvent such as 2-butanone, iso-butyl methyl ketone, acetone, dimethylsulfoxide, N,N-dimethylformamide or N-methylpyrrolidone. Since the process is to be performed at a temperature in the range of 50 ° C. to 150° C. it will be appreciated by those skilled in the art that the process may optionally be performed under pressure in order to achieve the desired reaction temperature with solvents which have a boiling point below the desired reaction temperature.

The process of the invention has the following advantages. The reaction times are more rapid than the reactions known in the prior art and therefore the process is less costly to run. In addition the process gives higher yields and the product is of a higher purity than previously s disclosed processes for the preparation of the compound of formula 1. Further, the process is consistently reproducible and robust.

In the process where a melt is used greater purity is achieved and the reaction is more volume effective, i.e the same reaction vessel will produce greater yields.

In another aspect the present invention provides a process for the preparation of a compound of formula I 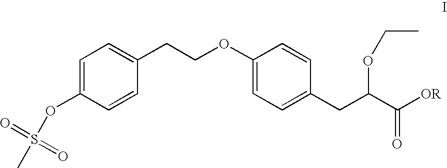
in which R represents H which comprises reacting a compound of formula II 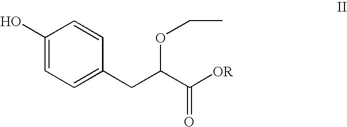
in which R represents an acid protecting group with a compound of formula III 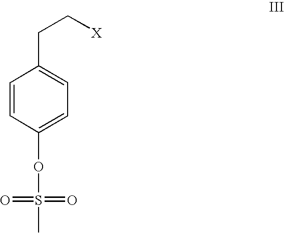
wherein X is a suitable leaving group in the presence of a base in solid form and a phase transfer catalyst at a temperature in the range 50° C. to 150° C. to give a compound of formula I in which R is an acid protecting group and then removing the protecting group to give a compound of formula I in which R is H.

In a preferred aspect the process provides the S-enantiomer of the compound of formula I in which R is H by using the S-enantiomer of the compound of formula II in which R represents H or an acid protecting group followed by hydrolysis when R is an acid protecting group.

The compound of formula I in which R is H may be purified by recrystallisation. Suitable recrystallisation solvents include one or more of the following ethanol, water, isopropylacetate, isopropanol, isooctane and toluene.

The invention is illustrated by the following non-limiting examples.

Abbreviations

EtOAc=ethyl acetate
HPLC=high-pressure liquid chromatography
i-PrOAc=isopropyl acetate
PEG=polyethylene glycol
MEK=Methy ethyl ketone or butan-2-one
MIBK=Methyl isobutyl ketone or 3 methylbutan-2-one
EtOH=ethanol
   Preparation of Starting Material

2-(4-Methanesulfonyloxyphenyl)ethylmethanesulfonate was prepared as described in WO99/62872.

### EXAMPLE 1

### Ethyl (S)-2-ethoxy-3-14-(2-{4-methanesulfonyloxyphenyl}ethoxy)phenyl] propanoate

2-(4-Methanesulfonyloxyphenyl)ethylmethanesulfonate (298.5 g, 1.01 mol), ethyl (2S)-2-ethoxy-3-(4-hydroxyphenyl)propanoate (96.7 g, 406 mmol) and PEG-400 (32.5 g, 81 mmol) were melted together at 110° C. Na₂CO₃ (56.8 g, 536 mmol) was then added under vigorous stirring. The reaction was continued at this temperature for 5.5 hours. (HPLC control of conversion >95%). The mixture was then cooled to 45 ° C. Acetone (500 ml) was added. The mixture was stirred until all organic material was dissolved. The inorganic salts were then filtered off. The salts were washed twice with acetone (2×300 ml). The acetone solution of the title compound was used directly in the next step.

### (S)-2-Ethoxy-3-14-(2-{4-methanesulfonyloxyphenyl}ethoxy)phenyl] propanoic acid

To the acetone solution containing ethyl (S)-2-ethoxy-3-[4-(2-{4-methanesulfonyl-oxyphenyl}ethoxy)phenyl]propanoate was charged water (200 ml). Under vigorous stirring concentrated NaOH (aq) (34 ml, 568 mmol) was charged. The solution was then heated to +30 ° C. and the reaction continued for 6 hours (HPLC control conversion >99%) under vigorous stirring. The reaction was quenched with EtOAc (140 ml). When the pH was 11 evaporation of organic solvents was started at about 50 ° C. under vacuum. When all volatile solvents were removed (750 ml solution remaining in the reactor), water (100 ml) was charged and the distillation continued until 520 ml of the solution remained in the reactor. The solution was cooled to 20 ° C. and water (280 ml) was charged. The solution was then extracted 3 times with EtOAc (2×600 ml, 1×400 ml). The pH was then corrected to 2-2.5 using 25 % H₂SO₄. The solution was heated to 50° C. and remaining EtOAc was distilled of under vacuum. The evaporation was terminated when water started to distil. The acidic water solution was then extracted with toluene (605 ml) at 50° C. The toluene phase was washed with water (380 ml) at 50 ° C. The toluene solution was then cooled to 20° C. over about 1 hour. At 20° C. the solution was seeded with (S)-2-ethoxy-3-[4-(2-{4-methanesulfonyloxyphenyl}-ethoxy)phenyl]propanoic acid. The slurry formed was then cooled to 8° C. and left crystallising over night. The product was filtered and washed with 8° C. toluene (160 ml).

### EXAMPLE 2

### 2-(4-Methanesulfonyloxyphenyl)ethylmethanesulfonate

2-(4-Hydroxyphenyl)ethanol (20.0 g, 143.7 mmol) was dissolved in 2-butanone (MEK, 200 ml) and triethylamine (44.3 ml, 316.2 mmol). The mixture was cooled to 3° C. after a clear solution was obtained. Methanesulfonyl chloride (23.4 ml, 301.8 mmol) was then added during about 15 minutes keeping the temperature below 17° C. The conversion was checked 25 minutes after all the methanesulfonyl chloride was added. The slurry was cooled to 6° C. and the salts formed were filtered off and washed with +8° C. 2-butanone (MEK, 50 ml). The MEK-solution of 2-(4-methanesulfonyloxyphenyl)-ethylmethanesulfonate was then used in the following step.

### Ethyl (S)-2-ethoxy-3-[4-(2-{4-methanesulfonyloxyphenyl}ethoxy)phenyl] propanoate

Ethyl (S)-2-ethoxy-3-(4-hydroxyphenyl)propanoate (10.0 g, 41.5 mmol) was dissolved in the MEK solution containing 2-(4-methanesulfonyloxyphenyl)ethylmethanesulfonate (ca 105 ml, 60.2 mmol). When a homogenous solution was formed, PEG-400 (4.0 g, 40 weight % to ethyl (S)-2-ethoxy-3-(4-hydroxyphenyl)propanoate ) and K₂CO₃ (8.67 g, 62.2 mmol) were added under vigorous stirring. The mixture was allowed to react at reflux, 79° C. for about 6 hours under vigorous stirring. The reaction mixture was cooled to 26° C. and MEK (10 ml) was added. Water (50 ml) was added and the phases were separated. The organic layer was washed once more with water (20 ml). The organic phase was then used in the following step.

### (S)-2-Ethoxy-3-[14-(2-{4-methanesulfonvioxyphenyl}ethoxy)phenyl] propanoic acid

To the 2-butanone solution (total volume about 50 ml) of ethyl (S)-2-ethoxy-3-[4-(2-{4-methanesulfonyloxyphenyl}ethoxy)phenyl]propanoate (7.55 g, 17.2 mmol) was added acetone (25 ml), water (5 ml) and NaOH (3M, 7.5 ml). Acetone (10 ml) was added and the mixture was heated to 30° C. After about 3.5 hours the mixture was cooled to 25° C. and EtOAc (20 ml) was added. The mixture was then evaporated under vacuum, removing the volatile solvents. During the evaporation a further portion of water (40 ml) was added. EtOAc (40 ml) was added and the mixture extracted. The phases were separated and the water layer was washed once more with EtOAc (15 ml). Toluene (30 ml) was then added and the pH of the mixture was adjusted to 2.1 with H₂SO₄ (conc). The layers were separated and the water layer extracted once more with toluene (8 ml). The combined organic layers were washed once with water (8 ml). The toluene solution was then evaporated down to a volume of 20 ml. The solution was seeded and crystallised while cooling. Toluene (6 ml) was added to make the slurry more mobile. The product was filtered and washed once with cold toluene (10 ml). The crystals were then dried under vacuum.

### EXAMPLE 3

2-(4-Methanesulfonyloxyphenyl)ethylmethanesulfonate (1.64 g, 5.41 mmol) was dissolved in 2-butanone (10.0 ml) at 22° C. Ethyl (2S)-2-ethoxy-3-(4-hydroxyphenyl)propanoate (1.0 g, 4.16 mmol), 18-Crown-6 (0.06 g, 0.21 mmol) and K₂CO₃ (0.81 g, 5.82 mmol) added. The mixture was boiled under reflux for 4 hours. A conversion of 93% was reached.

### EXAMPLE 4

2-(4-Methanesulfonyloxyphenyl)ethylmethanesulfonate (3.09g, 10.5 mmol), ethyl (S)-2-ethoxy-3-(4-hydroxyphenyl)propanoate (1.00 g, 4.20 mmol) and dibenzo-18-Crown-6 (0.15 g, 0.42 mmol) were mixed together. The mixture was heated to 90° C. and K₂CO₃ (1.25 g, 5.46 mmol) was added. The reaction was allowed to proceed at 110° C. for 4 hours. A conversion of 91 % was reached.

### EXAMPLE 5

A mixture of 2-(4-methanesulfonyloxyphenyl)ethylmethanesulfonate (296.5 g/1.6 mol eq), ethyl (S)-2-ethoxy-3-(4-hydroxyphenyl)propanoate (150 g/1.00 mol eq), PEG-400 (50.36 g/0.2 mol eq/0.30 rel vol), Na₂CO₃ (88.74 g/1.33 mol eq) and water (300.0 g/26.5 mol eq/2.0 rel vol) was boiled under reflux with vigorous stirring for about 3-7 h. The mixture was cooled to Tᵢ=85-95° C. and the phases are separated. Acetone (316.0 g/8.64 mol eq/2.7 rel vol, T_{b}=56.2° C.) was charged to the organic phase at Tᵢ<55° C. Cooling was continued down to Tᵢ=25° C.

The acetone solution containing ethyl (S)-2-ethoxy-3-[4-(2-{4-methanesulfonyloxy-phenyl}ethoxy)phenyl] propanoate was kept at Tᵢ=25° C. in the reactor. LiOHxH₂O powder (34.3 g/1.3 mol eq) dissolved in water (600.0 g/4.0 rel vol) was charged continuously over 30 minutes under vigorous stirring at 25±5° C. The reaction was continued for 1-3 hours. The reaction was quenched by addition of EtOAc (40.5 g/0.73 mol eq/0.3 rel vol) at Tᵢ=25±5° C. Acetone, EtOH and EtOAc was removed from the solution by vacuum evaporation at Tᵢ<35° C. The solution was washed once with isopropyl acetate (5.0 rel vol/750.0 ml) Tᵢ=35° C. The water layer was evaporated down to a volume of 600 ml /4.0 rel vol prior to crystallisation and also to remove residues of EtOAc.

Acetic acid (629.4 g/600 ml/4.0 rel vol) was charged to the solution under good stirring at 25° C. and a clear solution should be formed. The solution was seeded using (S)-2-ethoxy-3-[4-(2-{4-methanesulfonyloxyphenyl}ethoxy)phenyl] propanoic acid (0.75 g ). The solution was cooled to 20° C. A mixture of water (600 g/600 ml/4 rel vol) and sulphuric acid (27.6 g/15.0 ml/0.1 rel vol) was charged to the slurry keeping the temperature at 20° C. After the water charge, pH was checked and adjusted to 2-2.5 using H₂SO₄ or LiOHxH₂O. The slurry was cooled to −4° C. The slurry was left at −4° C. for 19 hours. The slurry was filtered and washed with water (2×450 ml/2×3.0 rel vol). (S)-2-Ethoxy-3-[4-(2-{4-methanesulfonyloxyphenyl}ethoxy)phenyl] propanoic acid was dried under vacuum at 40° C.

1. A process for the preparation of a compound of formula I 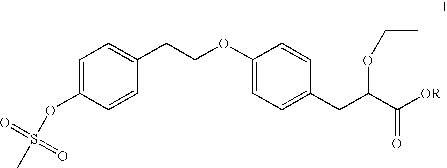
in which R represents H or an acid protecting group which comprises reacting a compound of formula II 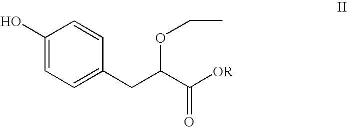
in which R is as previously defined with a compound of formula III 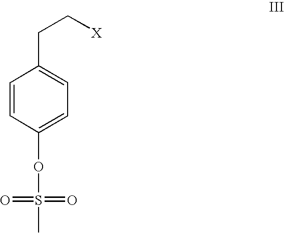
wherein X is a suitable leaving group in the presence of a base and a phase transfer catalyst at a temperature in the range 50° C. to 150° C.:

2. A process according to claim 1, wherein the process is performed in the presence of an aqueous solution of a base and a phase transfer catalyst.

3. A process according to claim 1, wherein the process is performed in the presence of a base in solid form and a phase transfer catalyst.

4. A process according to claim 1, further comprising removing an acid protecting group to produce a compound of formula I in which R is H.

5. A process according to claim 4, in which the acid protecting group is removed by hydrolysis.

6. A process according to claim 3 or 4, in which the process is carried out as a melt.

7. A process according to claim 1, in which R is a (1-4C)alkyl group.

8. A process according to claim 1, in which X is halo, an optionally substituted phenylsulfonyloxy group or an alkylsulphonyloxy group.

9. A process according to claim 1, in which the base is selected from carbonates, hydrogen carbonates or hydroxides of alkali metals.

10. A process according to claim 1, in which the phase transfer catalyst is a crown ether, a polyethylene glycol or a quaternary ammonium salt.

11. A process according to claim 1 in which the process is carried out in the presence of a suitable solvent for the compounds of formulae II and III.

12. A process according to claim 11, in which the solvent is selected from 2-butanone, iso-butyl methyl ketone, acetone, dimethylsulfoxide, NN-dimethylformamide, or N-methylpyrrolidone.

13. A process according to claim 1, in which the compound of formula I is the S enantiomer.

14. A process for the preparation of a compound of formula I 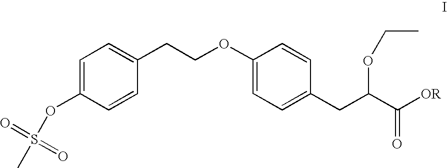
in which R represents H which comprises reacting a compound of formula II 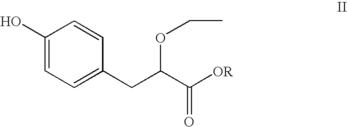
in which R represents an acid protecting group with a compound of formula III 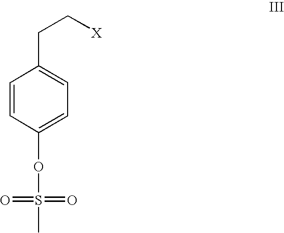
wherein X is a suitable leaving group in the presence of a base in solid form and a phase transfer catalyst at a temperature in the range 50° C. to 150° C. to give a compound of formula I in which R is an acid protecting group and then removing the protecting group to give a compound of formula I in which R is H.
